(19) United States
(12) Patent Application Publication
(10) Pub. No.: US 2011/0003356 A1
(43) Pub. Date: Jan. 6, 2011

(54) PROCESS FOR PRODUCTION OF XYLITOL

(75) Inventors: Manish Jain, Pune (IN); K.V. Satyanarayana, Pune (IN); Ashok Kumar Dubey, Pune (IN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TATA Chemicals Limited, Mumbai (IN)

(21) Appl. No.: 12/922,074

(22) PCT Filed: Jan. 9, 2009
(86) PCT No.: PCT/IN2009/000027

§ 371 Date: Sep. 10, 2010

(30) Foreign Application Priority Data
Mar. 10, 2008 (IN) 481/MUM/208

(51) Int. Cl. C12P 7/18

(52) U.S. Cl. 435/158

(57) ABSTRACT

The invention provides a process for producing xylitol from xylose by a yeast strain capable of converting the xylose to xylitol comprising independently growing the yeast strain in a medium; transferring the yeast strain from the medium to a feed solution, the feed solution comprising of xylose in water, and separating the xylitol from said feed solution.

The invention relates to the production of xylitol. More particularly the invention relates to the production of xylitol by microbial conversion of xylose in water.

### DESCRIPTION OF RELATED ART

Xylitol is a five-carbon sugar alcohol with significant medical and commercial applications and has been widely used as an alternative low calorie sweetener. Xylitol occurs naturally in many fruits and vegetables and also as a natural intermediate product in glucose metabolism of humans and other animals as well as in the metabolism of several plants and microorganisms.

Xylitol has numerous advantages including the same sweetness as sucrose but with one-third fewer calories and no unpleasant aftertaste. In addition its negative heat of dissolution imparts a cool and refreshing sensation in the oral cavity, making it a popular sweetener for candies and sweets. Xylitol also finds favor with diabetic patients as it is metabolized independently from insulin in the human body. Xylitol has also been shown to have therapeutic properties and reportedly builds immunity, fights against chronic degenerative diseases, is anti-aging, and has no known toxic levels.

In addition to its use in the food, pharmaceutical and oral health products, xylitol also finds commercial applications in plywood industries and can replace phenolic resins because of its adhesive properties.

The importance of xylitol and its application in different industries requires methods for maximum production of this sugar alcohol in an efficient manner. As xylitol is present in low concentrations in vegetables or fruits its extraction is uneconomical from these sources. Commercially xylitol is produced by chemical reduction of xylose, which is a hydrolysate of hemicellulose present in woods, rice straw, millet, etc. An example of this process is described in the U.S. Pat. No. 4,008,285, in which the production of xylitol on a commercial scale is carried out by acid hydrolysis of pentosan-containing raw materials such as wood, corncobs, straw, bran, and cottonseed hulls. The hydrolysis of xylitol is usually carried out using Raney nickel catalyst (Ni/Al₂O₃) at high temperature and pressure. One limitation of the chemical process is the difficulty of separation and purification of xylose or xylitol from hydrolysates of other polymer sugars derived from hemicellulose fractions. Multistep separation techniques, including mechanical filtration and chromatography are required to obtain pure xylitol. These processes adversely affect the cost of production for a yield in the range of 50-60%. Furthermore, such processes involve high temperature and high-pressure associated risks. Waste disposal due to use of acid or alkali is another major concern that is associated with chemical production of xylitol. These factors make the chemical methods for the routine production of xylitol difficult, expensive and inefficient.

On the other hand biotechnological processes have the advantages of selective conversion with high yield and less toxic/nontoxic byproducts where xylose is converted into xylitol by microorganisms using NAD(P)H- dependent xylose reductase (XR).

Several biological processes using microorganisms for the production of xylitol from xylose are known. A number of yeast including those of the species Pichia, Candida, Hansenula and Kluyveromyces, are capable of reducing xylose to xylitol as an initial step in their xylose metabolism/bioconversion. The production of xylitol using microorganisms has been carried out by batch, fed batch and cell recycling methods.

The cost of microbial production of xylitol depends on a number of factors including the cost of fermentation medium, maintenance of fermentation conditions and cost of recovery of xylitol from the fermentation medium. Microbial methods currently available for the production of xylitol use medium containing xylose along with nutrients that facilitate cell growth. Nutrients that are added are either complex organic compounds like peptone, yeast extract etc. or chemical compounds.

Since these nutrients are expensive they increase the cost of production. Moreover, by using such a medium it is difficult to efficiently and selectively remove byproducts, medium ingredients and substances derived from microorganism, from the fermentation medium and recover xylitol. This further increases the cost of xylitol production.

In view of these difficulties faced in the production of xylitol, there is a need to develop an easy, cost effective and ecologically friendly method for production of xylitol.

### SUMMARY

The invention relates to a process for producing xylitol from xylose by a yeast strain capable of converting the xylose to xylitol comprising independently growing the yeast strain in a medium; transferring the yeast strain from the medium to a feed solution, the feed solution comprising of xylose in water, and separating the xylitol from said feed solution.

The invention relates to a process for producing xylitol from xylose by a yeast strain capable of converting the xylose to xylitol comprising independently growing the yeast strain in a medium; transferring the yeast strain from the medium to a feed solution, the feed solution comprising of xylose in water; recovering the xylitol from said feed solution; and recycling the yeast strain at least once, the recycling comprising transferring the yeast strain to another feed solution; the other feed solution comprising of xylose in water.

The invention relates to a process for producing xylitol from xylose by a yeast strain capable of converting the xylose to xylitol comprising growing the yeast strain in a medium including xylose to obtain xylitol; recovering xylitol from medium and transferring the yeast strain from the medium to a feed solution, the feed solution comprising of xylose in water, and separating the xylitol from said feed solution.

The invention relates to a process for producing xylitol from xylose by a yeast strain capable of converting the xylose to xylitol comprising growing the yeast strain in a medium including xylose to obtain xylitol; recovering xylitol from medium and transferring the yeast strain from the medium to a feed solution, the feed solution comprising of xylose in water, recovering xylitol from the feed solution and recycling the yeast strain at least once, the recycling comprising transferring the yeast strain to another feed solution; the other feed solution comprising of xylose in water.

### BRIEF DESCRIPTION OF ACCOMPANYING DRAWINGS

The accompanying drawings illustrate the preferred embodiments of the invention and together with the following detailed description serve to explain the principles of the invention.

FIG. 1 illustrates shake flask experimental results for xylitol production using C. tropicalis ATCC 13803 at 30° C., 250 rpm and pH 6.0.

FIG. 2: illustrates the experimental results of % Xylitol Production using C. tropicalis ATCC 13803 at 30° C., 250 rpm and pH 6.0.

FIG. 3: illustrates the experimental results of cell recycling experiment for Xylitol Production from Xylose (100 g/L) using C. tropicalis ATCC 13803 at 30° C., 215 rpm.

FIG. 4: Cell recycling experiment for Xylitol Production from Xylose (150 g/L) using C. tropicalis ATCC 13803 at 30° C., 215 rpm.

Table 1: Summary of results obtained in cell recycling experiments with different xylose concentrations.

### DETAILED DESCRIPTION OF PREFERRED EMBODIMENTS

To promote an understanding of the principles of the invention, reference will be made to the embodiment illustrated in the drawing and specific language will be used to describe the same. It will nevertheless be understood that no limitation of scope of the invention is thereby intended, such alterations and further modifications in the described method and such further applications of the principles of the inventions as illustrated therein being contemplated as would normally occur to one skilled in art to which the invention relates.

It will be understood by those skilled in the art that the foregoing general description and the following detailed description are exemplary and explanatory of the invention and are not intended to be restrictive thereof.

The invention provides a method for microbial production of xylitol from a solution of xylose in water. More particularly the invention relates to the production of xylitol from a solution of xylose in water by a yeast strain capable of converting xylose to xylitol.

The invention relates to the process of production of xylitol from a solution of xylose in water by yeast wherein the yeast strain is inoculated into a solution of xylose in water to produce xylitol.

In accordance with an aspect, a process for producing xylitol from xylose by a yeast strain capable of converting the xylose to xylitol is disclosed. The process includes independently growing the yeast strain in a medium and transferring the yeast strain from the medium to a feed solution. The feed solution comprises of xylose in water. Xylitol produced in the feed solution is recovered.

The yeast strain is grown independently in a nutrient rich medium, which includes growing the yeast strain in a nutrient rich growth medium till required cell mass is achieved. Yeast cells so grown are separated from the growth medium and transferred to a feed solution of xylose in water.

In accordance with an aspect, a process for producing xylitol from xylose by a yeast strain capable of converting the xylose to xylitol is disclosed. The process includes independently growing the yeast strain in a medium and transferring the yeast strain from the medium to a feed solution. The feed solution comprises of xylose in water. Xylitol produced in the feed solution is recovered and the yeast strain is recycled at least once. The recycling comprises transferring the yeast strain to another feed solution that comprises xylose in water.

The yeast strain is grown independently in a nutrient rich medium, which includes growing the yeast strain in a nutrient rich growth medium till required cell mass is achieved. Yeast cells so grown are separated from the growth medium and transferred to a feed solution comprising of xylose in water. Xylitol produced is recovered from the feed solution and the yeast strain is recycled at least once by introducing the yeast strain in another feed solution, comprising a solution of xylose in water.

In accordance with an aspect, a process for producing xylitol from xylose by a yeast strain capable of converting the xylose to xylitol comprising growing the yeast strain in a medium including xylose is disclosed. Xylitol produced is recovered from the medium and the yeast strain is transferred from the medium to a feed solution. The feed solution comprises of xylose in water. Xylitol produced is recovered from the feed solution.

The yeast strain is grown independently in a nutrient rich medium including xylose, which includes growing the yeast strain in a nutrient rich growth medium including xylose till required cell mass is achieved. Yeast cells so grown are separated from the growth medium and transferred to a feed solution of xylose in water. Xylitol is recovered from both medium.

In accordance with an aspect, a process for producing xylitol from xylose by a yeast strain capable of converting the xylose to xylitol comprising growing the yeast strain in a medium including xylose is disclosed. The process includes growing the yeast strain in a medium including xylose and transferring the yeast strain from the medium to a feed solution. Xylitol produced is recovered from the medium. The feed solution comprises of xylose in water. Xylitol produced in the feed solution is recovered and the yeast strain is recycled at least once. The recycling comprises transferring the yeast strain to another feed solution that comprises of xylose in water.

The yeast strain is grown in a nutrient rich medium including xylose, which includes growing the yeast strain in a nutrient rich growth medium till required cell mass is achieved and xylitol is produced. Yeast cells so grown are separated from the medium and transferred to a feed solution of xylose in water. Xylitol produced is recovered from the feed solution and the yeast strain is recycled at least once by introducing the yeast strain in a medium containing a solution of xylose in water.

In accordance with an aspect, a process for producing xylitol from xylose by a yeast strain capable of converting the xylose to xylitol is disclosed. The process includes independently growing the yeast strain in a medium to which nutrients are gradually added and transferring the yeast strain from the medium to a feed solution. The feed solution comprises of xylose in water. Xylitol produced in the feed solution is recovered.

The yeast strain is grown independently in a nutrient rich medium, in which nutrients are gradually added, which includes growing the yeast strain in a growth medium gradually supplemented by nutrients till required cell mass is achieved. Yeast cells so grown are separated from the growth medium and transferred to a feed solution of xylose in water.

In accordance with an aspect, a process for producing xylitol from xylose by a yeast strain capable of converting the xylose to xylitol is disclosed. The process includes independently growing the yeast strain in a medium to which nutrients are gradually added and transferring the yeast strain from the medium to a feed solution. The feed solution comprises of xylose in water. Xylitol produced in the feed solution is recovered and the yeast strain is recycled at least once. The recycling comprises transferring the yeast strain to another feed solution that comprises of xylose in water.

The yeast strain is grown independently in a nutrient rich medium, in which nutrients are gradually added, which includes growing the yeast strain in a growth medium gradually supplemented by nutrients till required cell mass is achieved. Yeast cells so grown are separated from the growth medium and transferred to a feed solution of xylose in water. Xylitol produced is recovered from the feed solution and the yeast strain is recycled at least once by introducing the yeast strain in another feed solution that comprises of xylose in water.

In accordance with an aspect, a process for producing xylitol from xylose by a yeast strain capable of converting the xylose to xylitol comprising growing the yeast strain in a medium including xylose, where nutrients and xylose are gradually added is disclosed. Xylitol produced is recovered from the medium and the yeast strain is transferred from the medium to a feed solution. The feed solution comprises of xylose in water. Xylitol produced is recovered from the feed solution.

The yeast strain is grown in a nutrient rich medium, in which nutrients and xylose are gradually added, which includes growing the yeast strain in a growth medium gradually supplemented by nutrients and xylose till required cell mass is achieved. Yeast cells so grown are separated from the medium and transferred to a feed solution of xylose in water. Xylitol is recovered from both mediums.

In accordance with an aspect, a process for producing xylitol from xylose by a yeast strain capable of converting the xylose to xylitol comprising growing the yeast strain in a medium including xylose where nutrients and xylose are gradually added is disclosed. The process includes growing the yeast strain in a medium including xylose and transferring the yeast strain from the medium to a feed solution. Xylitol produced is recovered from the medium. The feed solution comprises of xylose in water. Xylitol produced in the feed solution is recovered and the yeast strain is recycled at least once. The recycling comprises transferring the yeast strain to another feed solution that comprises of xylose in water.

The yeast strain is grown in a nutrient rich medium, in which nutrients and xylose are gradually added, which includes growing the yeast strain in a growth medium gradually supplemented by nutrients and xylose till required cell mass is achieved and xylitol is produced. Xylitol produced is recovered from the medium. Yeast cells so grown are separated from the medium and transferred to a feed solution of xylose in water. Xylitol produced is recovered from the feed solution and the yeast strain is recycled at least once by introducing the yeast strain in a medium containing a solution of xylose in water.

It is preferred that the nutrient rich media used promotes maximum cell growth of the yeast strain. In accordance with embodiments of the invention, the media used for the cell growth may be complex or chemically defined. The media used for cell growth may also contain xylose such that xylitol is produced during the cell growth stage.

The growth medium may be a complex medium containing complex organic compounds including but not limited to peptone and yeast extract as nitrogen source. Glucose, xylitol or any other carbon source may be used separately or in combination with each other. The glucose and xylose may be autoclaved separately and added to the medium.

In accordance with an aspect, the pH of the medium is preferably maintained at 6.0 and cell concentration is preferably monitored turbidometrically at 600 nm.

The separation of the yeast cells from medium or feed solution may be carried out by any separation method including but not limited to filtration and centrifugation. Xylitol may be recovered from the media or feed solution by common purification process.

The yeast strain used for xylitol production should have the ability to convert xylose to xylitol. In accordance with a preferred embodiment, the yeast may be of genus Candida. More preferably the strain of the genus may be Candida tropicalis or its mutants. Most preferably the strain may be Candida tropicalis ATCC 13803.

In accordance with an embodiment the process for production of xylitol is described wherein xylitol is produced by the conversion of xylose in water by a yeast strain capable of converting the xylose to xylitol that is grown independently in a nutrient rich medium. The yeast strain is inoculated onto an agar plate containing growth medium and allowed to grow till colonies develop. Colonies of yeast strain are then picked up from the plate, inoculated into an inoculation medium and allowed to grow for the required inoculation period. Required amount of inoculum is added to a nutrient rich medium and yeast strain allowed to grow till the required cell mass is achieved. Yeast strain is then separated from the nutrient rich medium, transferred to a feed solution of xylose in water and incubated for required incubation period for the production of xylitol. Xylitol is recovered from the feed solution by common purification method.

In accordance with an aspect, a process for production of xylitol is described wherein a yeast strain capable of converting the xylose to xylitol is independently grown in a nutrient rich medium and is recycled in a feed solution of xylose in water. Yeast is inoculated onto an agar plate containing growth medium and allowed to grow till colonies develop. Colonies of yeast are picked up from the plate, inoculated into an inoculation medium and grown for the required incubation period. Required amount of inoculum is added to the nutrient rich growth medium and incubated till the required cell mass is achieved. The yeast strain is separated from the nutrient rich growth medium, and added to a feed solution of xylose in water and incubated for the required incubation period. The yeast strain is separated again, reintroduced into a feed solution and incubated for the required incubation period. These steps are repeated at least once. The feed solution from each cell recycling stage is collected and xylitol is recovered by common purification process.

In accordance with an embodiment the process for production of xylitol is described wherein xylitol is produced by conversion of xylose in water by a yeast strain capable of converting the xylose to xylitol that is grown independently in a nutrient rich medium including xylose. The yeast strain is inoculated onto an agar plate containing growth medium and allowed to grow till colonies develop. Colonies of yeast strain are then picked up from the plate, inoculated into an inoculation medium and allowed to grow for the required inoculation period. Required amount of inoculum is added to a nutrient rich medium containing xylose and yeast strain allowed to grow till the required cell mass is achieved. Xylitol is recovered from the medium. Yeast strain is then separated from the nutrient rich medium, transferred to a feed solution of xylose in water and incubated for required incubation period for the production of xylitol. Xylitol from the feed solution is recovered by common purification method.

In accordance with an embodiment a process for production of xylitol is described wherein yeast strain capable of converting the xylose to xylitol is grown in a growth medium including xylose and recycled in a solution of xylose in water. Yeast is inoculated onto an agar plate containing growth medium and allowed to grow till colonies develop. Colonies of yeast are picked up from the plate, inoculated into an inoculation medium and allowed to grow. Required amount of inoculum is added to the nutrient rich growth medium including xylose and incubated till the required cell mass is achieved and xylose is converted into xylitol. The yeast strain is separated from the medium, added to a feed solution of a solution of xylose in water and incubated for the required incubation period. Xylitol is recovered from the medium. After the required incubation period, the yeast strain is separated again and reintroduced into a feed solution of a solution of xylose in water and incubated for the required incubation period. These steps are repeated at least once. The feed solution from each stage cell recycling is collected and xylitol is recovered by common purification process.

In accordance with an embodiment the process for production of xylitol is described wherein xylitol is produced by conversion of xylose in water by a yeast strain capable of converting the xylose to xylitol that is independently grown in medium to which nutrients are gradually added. Yeast strain is inoculated onto an agar plate containing growth medium and allowed to grow till colonies develop. Colonies of yeast are then picked up, inoculated into an inoculation medium and allowed to grow. Required amount of inoculum is added to the growth medium that is gradually supplemented with nutrients. Yeast strain is allowed to grow till the required cell mass is obtained. The yeast strain is, then separated from the growth medium, added to a feed solution of xylose in water and allowed to react for required residence time. Xylitol is recovered from the feed solution by common purification process.

In accordance with an aspect, a process for production of xylitol is described wherein yeast strain capable of converting the xylose to xylitol is independently grown in a medium gradually supplemented with nutrients and recycled in a solution of xylose in water. Yeast cells are inoculated onto an agar plate containing growth medium and allowed to grow till colonies develop. Colonies of yeast were then picked up, inoculated into an inoculation medium and allowed to grow. Required amount of inoculum is added to the growth medium that is gradually supplemented with nutrients. Yeast strain is allowed to grow till the required cell mass is obtained. The yeast strain is separated from the growth medium, added to a feed solution of xylose in water and incubated for the required incubation period. After the required time, yeast strain is separated again and reintroduced into a feed solution of xylose in water and incubated. These steps are repeated at least once. The feed solution from each stage cell recycling is collected and xylitol is recovered by common purification process.

In accordance with an embodiment the process for production of xylitol is described wherein xylitol is produced by the conversion of xylose in water by a yeast strain capable of converting the xylose to xylitol that is grown in medium to which nutrients and xylose are gradually added. Yeast strain is inoculated onto an agar plate containing growth medium and allowed to grow till colonies develop. Colonies of yeast are then picked up, inoculated into an inoculation medium and allowed to grow. Required amount of inoculum is added to a growth medium that is gradually supplemented with nutrients and xylose. Yeast strain is allowed to grow till the required cell mass is obtained and xylose is converted to xylitol. Xylitol is recovered from the growth medium. The yeast strain is separated from the growth medium, added to a feed solution of xylose in water and incubated for required incubation period. Xylitol is recovered from the feed solution by common purification method.

In accordance with an embodiment, a process for production of xylitol is described wherein yeast strain capable of converting the xylose to xylitol is grown in a growth medium that is gradually supplemented with nutrients and xylose and recycled in a solution of xylose in water. Yeast strain is inoculated onto an agar plate containing growth medium and allowed to grow till colonies develop. Colonies of yeast were then picked up, inoculated into an inoculation medium and allowed to grow. Required amount of inoculum is added to the growth medium that is gradually supplemented with nutrients and xylose. Yeast strain is allowed to grow till the required cell mass is obtained and xylose is converted into xylitol. The yeast strain is then separated from the growth medium, added to a feed solution of xylose in water and allowed to react for required residence time. Xylitol is recovered from the growth medium. After the required time, cells are again separated and reintroduced into a solution of xylose in water and incubated. These steps are repeated at least once. The feed solution from each stage cell recycling is collected and xylitol is recovered by common purification process.

The temperature for xylitol production is preferably between the range of 26 to 30° C., with 30° C. being the preferred temperature.

In accordance with an aspect of the embodiment the yeast strain is agitated at a speed in the range of 150 to 250 rpm with the preferred speed being 215 rpm during xylitol production stage.

In accordance with an embodiment the cell recycling step is carried out at least once, preferably at least twice and most preferably three times in succession.

The concentration of xylose in water is in the range of 100 g/l to 200 g/l with the preferred concentration of xylose being 150 g/l.

In accordance with an aspect of an embodiment during the cell growth stage the cells are incubated at a temp range of 26 to 30° C., the preferred temperature being 30° C. The culture is agitated at a speed in the range of 150 to 250 rpm with the preferred speed being 250 rpm.

Xylitol is recovered from the feed solution by common purification process.

The following examples are provided to explain and illustrate certain preferred embodiments of the process of the invention.

### EXAMPLE 1

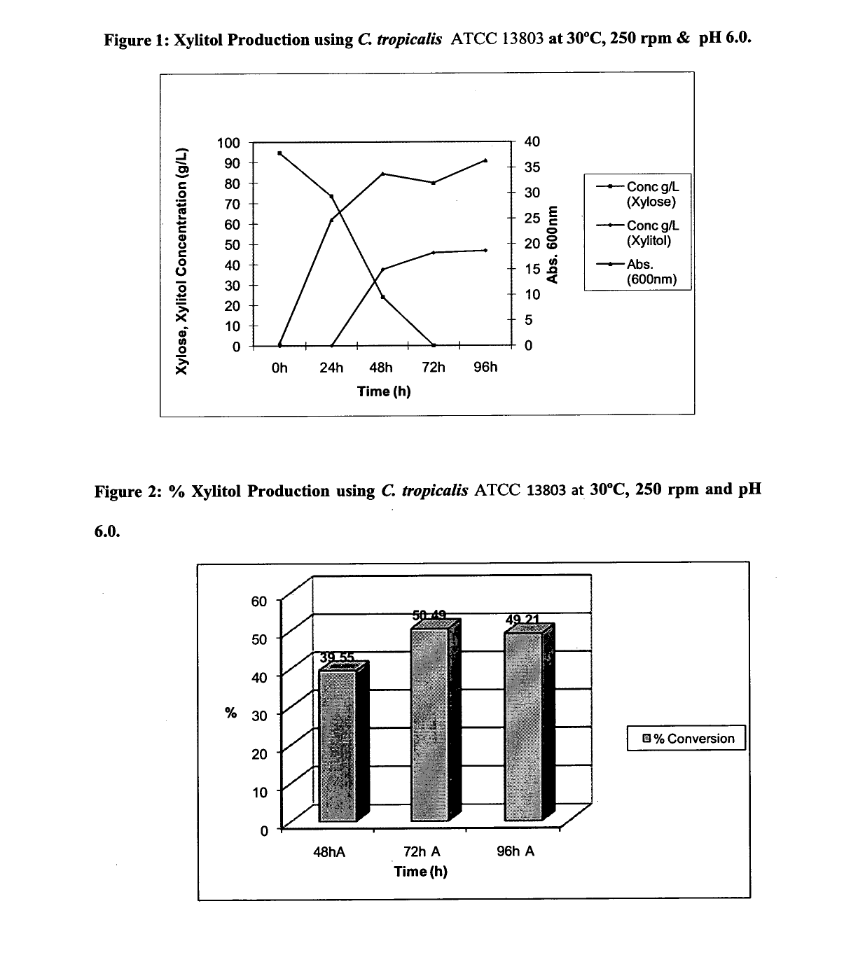

C. tropicalis ATCC 13803 was inoculated on YPX agar plate containing 50 g/L xylose, 10 g/L yeast extract, 20 g/L Bacto-peptone and 15 g/L agar. Yeast strain was then inoculated into an inoculation medium containing 25 g/L xylose, 10 g/L yeast extract, 20 g/L Bacto-peptone and 25 g/L glucose pH 6.0. 5% inoculum was added to the growth medium. Growth of yeast strain was carried out in a medium containing 100 g/L xylose, 10 g/L yeast extract, 20 g/L bacto-peptone and 30 g/L glucose, with pH adjusted to 6.0, at 30° C. and 250 rpm. Glucose and xylose were autoclaved separately and added to the media. Substrate and products were determined by HPLC equipped with Phenomenex RNM Carbohydrate Column (50×7.80 mm; 8 micron) and RID detector. Cell concentration was monitored turbidometrically at 600 nm. Xylitol production was 47.3 g/l (with xylitol yield of 0.5 g xylitol per gram xylose) at the end of 72 h (FIGS. 1 & 2).

### EXAMPLE 2

C. tropicalis ATCC 13803 was inoculated on YPX agar plate containing 50 g/L xylose, 10 g/L yeast extract, 20 g/L Bacto-peptone and 15 g/L agar. Cells of the yeast were then inoculated into an inoculation medium containing 25 g/L xylose, 10 g/L yeast extract, 20 g/L bacto-peptone and 25 g/L glucose at pH 6.0. 5% inoculum was added to the growth medium. Xylose and glucose were supplied at the rate of 100 g/l at 0 h, 72 h and 144 h and 30 g/l at 0 h, 10 g/l at 24 h and 5 g/l at 72 h and 144 h respectively in the growth medium. In addition to xylose and glucose the growth medium also contained 10 g/L yeast extract, 20 g/L bacto-peptone and its pH was adjusted to 6.0. The fermentation was carried out at a 30° C. and 250 rpm. Substrate and products were determined by HPLC equipped with Phenomenex RNM Carbohydrate Column (50×7.80 mm; 8 micron) and RID detector. Cell concentration was monitored turbidometrically at 600 nm. There was 50% conversion to xylitol in 196 h.

### EXAMPLE 3

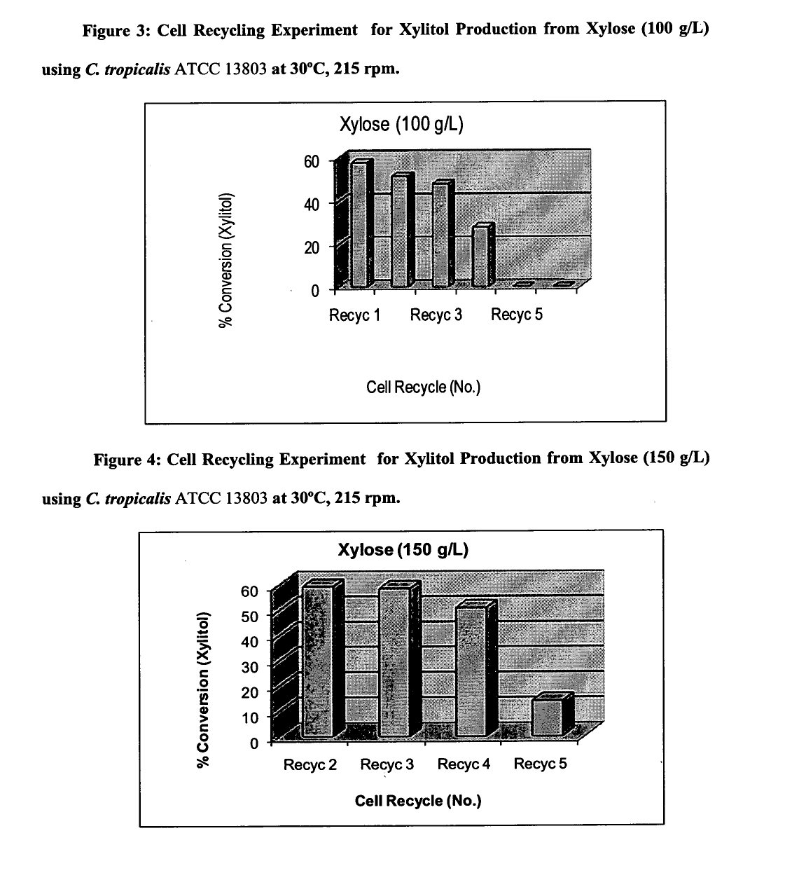

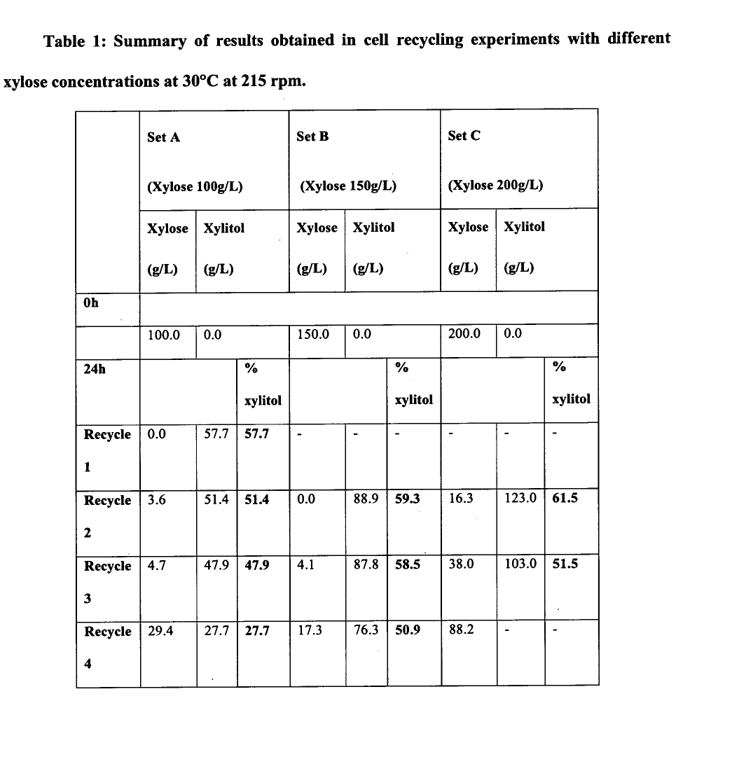

The growth medium containing yeast strain from batch or fed batch culture were centrifuged at 10,000 rpm at 4° C. for 15 minutes. After centrifugation strain was separated from medium by decanting the supernatant. The strain was recycled back to the flask containing xylose (100-200 g/L) in water and incubated at desired temperature (26-30° C.) and rpm (150-250) on the shaker. This procedure was repeated for 4 cycles and supernatant collected on each cycle were used for analysis purpose and purification of xylitol. Results with Xylose (150 g/L) to xylitol conversion were consistent (50-60%). (FIGS. 3 and 4 and Table 2).

1. A process for producing xylitol from xylose by a yeast strain capable of converting the xylose to xylitol comprising independently growing the yeast strain in a medium; transferring the yeast strain from the medium to a feed solution, the feed solution comprising of xylose in water, and separating the xylitol from said feed solution.

2. A process for producing xylitol from xylose by a yeast strain capable of converting the xylose to xylitol comprising independently growing the yeast strain in a medium; transferring the yeast strain from the medium to a feed solution, the feed solution comprising of xylose in water; recovering the xylitol from said feed solution; and recycling the yeast strain at least once, the recycling comprising transferring the yeast strain to another feed solution; the other feed solution comprising of xylose in water.

3. A process for producing xylitol from xylose by a yeast strain capable of converting the xylose to xylitol comprising growing the yeast strain in a medium including xylose to obtain xylitol; recovering xylitol from medium and transferring the yeast strain from the medium to a feed solution, the feed solution comprising of xylose in water, and separating the xylitol from said feed solution.

4. A process for producing xylitol from xylose by a yeast strain capable of converting the xylose to xylitol comprising growing the yeast strain in a medium including xylose to obtain xylitol; recovering xylitol from medium and transferring the yeast strain from the medium to a feed solution, the feed solution comprising of xylose in water, recovering xylitol from the feed solution and recycling the yeast strain at least once, the recycling comprising transferring the yeast strain to another feed solution; the other feed solution comprising of xylose in water.

5. A process for producing xylitol from xylose as claimed in claim 2 wherein the recycling step is carried out preferably two times and most preferably three times in succession.

6. A process as claimed in claim 1 wherein the yeast strain is of Candida Species.

7. A process as claimed in claim 6 wherein the yeast strain is preferably of Candida tropicalis or its mutants and most preferably Candida tropicalis ATCC 13803.

8. A process as claimed in claim 1 wherein the yeast strain is grown by fed batch culture.

9. A process as claimed in claim 3 wherein xylose is gradually added to the medium.

10. A process as claimed in claim 1 wherein the concentration of xylose in water is in the range of 100 g/l to 200 g/l, and preferably the concentration of xylose in water is 150 g/l.

11. A process as claimed in claim 1 wherein the medium is a complex or chemical medium.

12. A process as claimed in claim 1 wherein the pH of the medium is preferably maintained at 6.

13. A process as claimed in claim 1 wherein the temperature is preferably maintained in the range of 26 to 30 degrees.

14. A process for producing xylitol from xylose by a yeast strain capable of converting the xylose to xylitol, substantially as herein described.
